(19) United States
(12) Patent Application Publication
(10) Pub. No.: US 2022/0273411 A1
(43) Pub. Date: Sep. 1, 2022

(54) INTRARUMINAL DEVICE

(71) Applicants: Desmond Ian John MORROW, Auckland (NZ); David Anthony GILL, Auckland (NZ)

(72) Inventors: Desmond Ian John MORROW, Auckland (NZ); David Anthony GILL, Auckland (NZ)

(21) Appl. No.: 17/638,784

(22) PCT Filed: Aug. 31, 2020
(86) PCT No.: PCT/IB2020/058102

§ 371 Date: Feb. 25, 2022

(30) Foreign Application Priority Data
Aug. 29, 2019 (AU) 2019903173

(51) Int. Cl. A61D 7/00

(52) U.S. Cl. CPC A61D 7/00

(57) ABSTRACT

Provided is an intraruminal device comprising an elongate body or body assembly substantially impervious to rumen fluids, the body defining a barrel having a first end and a second end, a dose of an active agent within the body to be accessible to rumen fluid via substantially only the first outlet, a biasing arrangement within the body adapted to bias the active agent in the barrel towards the first end, and a first outlet at the first end comprising two or more apertures in the outlet, and a method of manufacturing the intraruminal device.

### RELATED APPLICATIONS

This application is a National Stage Application of International Application No. PCT/IB2020/058102, filed on Aug. 31, 2020, which claims priority to and the benefits of Australian Patent Application No. 2019903173, filed on Aug. 29, 2019, the entire contents of which are incorporated herein by reference.

### FIELD OF THE INVENTION

The present invention relates to an improved intra-ruminal device and method of making the device. In particular, the invention relates to an intra-ruminal device comprising a cap with multiple apertures.

### BACKGROUND TO THE INVENTION

Intra-ruminal devices are known, and are also termed variable geometry devices or Laby devices after Ralph H Laby who is recognised as developing the intraruminal device such as published in U.S. Pat. No. 4,671,789. The devices contain a matrix core material generally in the form of a stack of tablets that extrude out of the cap end of the devices. Such devices include a single hole at the cap end.

Such devices include a matric of tablets in the barrel. The tablets may contain a range of active agents in any order. Such devices suffer from a problem where adjacent capsules may coextrude out of the cap end aperture, which is not desired.

In this specification, where reference has been made to external sources of information, including patent specifications and other documents, this is generally for the purpose of providing a context for discussing the features of the present invention. Unless stated otherwise, reference to such sources of information is not to be construed, in any jurisdiction, as an admission that such sources of information are prior art or form part of the common general knowledge in the art.

It is an object of the present invention to provide an improved intraruminal devices or to at least provide the public with a useful choice.

### SUMMARY OF THE INVENTION

In a first aspect the invention relates to an intraruminal device comprising
an elongate body or body assembly substantially impervious to rumen fluids,
the body defining a barrel having a first end and a second end,
a dose of an active agent within the body to be accessible to rumen fluid via substantially only the first outlet,
a biasing arrangement within the body adapted to bias the active agent in the barrel towards the first end, and
a first outlet at the first end comprising two or more apertures in the outlet.

In a further aspect the present invention relates to a method of assembling an intra-ruminal device, the method comprising:

providing an intra-ruminal device comprising:
an elongate body or body assembly substantially impervious to rumen fluids, the body defining a barrel having a first end and a second end, and an opening at the first end,
at least one variable geometry device dependent from the body to assist rumen retention,
a dose of an active agent within the body to be accessible to rumen fluid via the first end,
a biasing arrangement within the body adapted to bias the active agent in the barrel towards the first end,
loading the active agent into the barrel,

attaching a cap located over the opening at the first end, the cap comprising an outlet in the top comprising at least 2 apertures.

In a further aspect the present invention relates to the use of an intra-ruminal device as described above in a ruminant.

The following embodiments may relate to any of the above aspects.

In one configuration the cap outlet comprises 2, 3, 4, 5, 6, 7, 8, 9 or 10 apertures, and suitable ranges may be selected from between any of these values.

In one configuration the cap outlet comprises 2, 3, 4, 5 or 6 apertures, and suitable ranges may be selected from between any of these values.

In one configuration the shape of the aperture is elliptical. However, other shapes, or combination of shapes including circles, slots and curves or a combination of shapes may be used to provide a symmetrical and uniform exposure of the core to the rumen fluids.

In one configuration the multiple apertures provide a uniform hydration across the face of the core to induce a linear erosion and extrusion.

In one configuration the largest diameter of an aperture is about 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8 or 5.0 mm, and suitable ranges may be selected from between any of these values.

In one configuration the total surface area of the apertures of the multiple-aperture cap of the present invention is within 1, 5, 10, 15, 20, 25, 20, 25 or 40% of a typical single aperture cap required to provide the desired payout of active, and suitable ranges may be selected from between any of these values.

In one configuration the total surface area of the apertures of the multiple-aperture cap of the present invention is about 10, 15, 20, 25, 30, 35, 40, 45, 50, 55 or 60 mm², and suitable ranges may be selected from between any of these values.

In one configuration the capsules have a pay-out (release rate) linearity of at least 0.940, 0.945, 0.950, 0.955, 0.960, 0.965, 0.970, 0.975, 0.980, 0.985, 0.990 or 0.995, and suitable ranges may be selected from between any of these values.

In one configuration the linearity measured over at least 80, 85, 90, 95, 100, 105, 110, 115 or 120 days, and suitable ranges may be selected from between any of these values.

In one configuration the capsules have pay-out (release rate) of about 0.3, 0.4, 0.5, 0.6, 0.78, 0.8, 0.9, 1.0, 1.1, 1.2, 1.3, 1.4 or 1.5 mm/day, and suitable ranges may be selected from between any of these values.

In one configuration the apertures of the multiple-apertured caps are the same diameter.

In one configuration the apertures of the multiple-apertured caps have a diameter that is within 2, 4, 6, 8, 10, 12, 14, 16, 18 or 20% of their average size.

In one configuration the body and the cap are held together under pressure, or heat and pressure.

In one configuration the intra-ruminal device is used in a group or herd of ruminants, each ruminant being administered an intra-ruminal device, and wherein the variable retention means remain attached to the body for at least the duration of the active agent payout period ensuring a continuous treatment period.

In one configuration the variable geometry retention means are designed to separate from the body after the payout period.

In one configuration the tablet core consists of two or more zones with each zone containing controlled-release tablets with similar or different release profiles/rates and contain similar of different individual or combination of actives ingredients.

In one configuration a portion of the core, one or more tablets, is designed to release an additional active to the main core to provide an exit dose capsule.

Preferably the barrel comprises a formulated core or stack of individual tablets.

In one configuration at least 80, 81, 82, 83, 84, 85, 86, 87, 88, 89, 90, 91, 92, 93, 94 or 95% of each tablet extrudes before the next tablet in the stack of tablet begins to extrude, and suitable ranges may be selected from between any of these values.

In one configuration adjacent tablet in the stack of tablet exhibit a reduction in co-extrusion.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7).

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

The term “comprising” as used in this specification means “consisting at least in part of”. When interpreting statements in this specification which include that term, the features, prefaced by that term in each statement, all need to be present but other features can also be present. Related terms such as “comprise” and “comprised” are to be interpreted in the same manner.

### BRIEF DESCRIPTION OF THE DRAWINGS

The invention will now be described by way of example only and with reference to the drawings in which:

FIG. 1 is a digital photograph of an opened capsule of design group (c).

FIG. 2 is a digital photographs of a capsule from design group (a) at day 18.

FIG. 3 is digital photograph of the bottom of the tank plate.

FIG. 4A is a digital photograph of a tablet at day 21, split in two.

FIG. 4B is a digital photograph of a tablet at day 21, split in two.

FIG. 4C is a digital photograph of a tablet with five apertures at day 21, split in two.

FIG. 4D is a digital photograph of a tablet with four apertures at day 21, split in two.

FIG. 5 is a digital photograph of a capsule from test group E at day 24.

FIG. 6A is a graph showing the pay-out profile for test group H.

FIG. 6B is a graph showing the pay-out profile for test group I.

FIG. 6C is a graph showing the pay-out profile for test group J.

FIG. 6D is a graph showing the pay-out profile for test group K.

FIG. 7A is a graph showing pay-output profiles for mid and end pulsatile capsules with single (test group N) or multi-hole (test group O) orifices. The shaded regions correspond to where the pulse tablets are extruded from the device.

FIG. 7B is a graph showing pay-output profiles start, mid and end pulsatile capsules with single (test group P) or multi-hole (test group R) orifices. The shaded regions correspond to where the pulse tablets are extruded from the device.

FIG. 8 is a perspective view of an intra-ruminal device (plunger not shown).

### DETAILED DESCRIPTION OF THE INVENTION

Described is an intra-ruminal device that comprises an elongate body or body assembly substantially impervious to rumen fluids. The body defines a barrel having a first end and a second end, with an opening at the first end. The device includes at least one variable geometry device dependent from the body to assist rumen retention. Within the body is a dose of an active agent accessible to rumen fluid via the first end. Also within the body is a biasing arrangement that is adapted to bias the active agent in the barrel towards the first end. When assembled, the device includes a cap having multiple apertures.

### 1. Intra-Ruminal Device Body

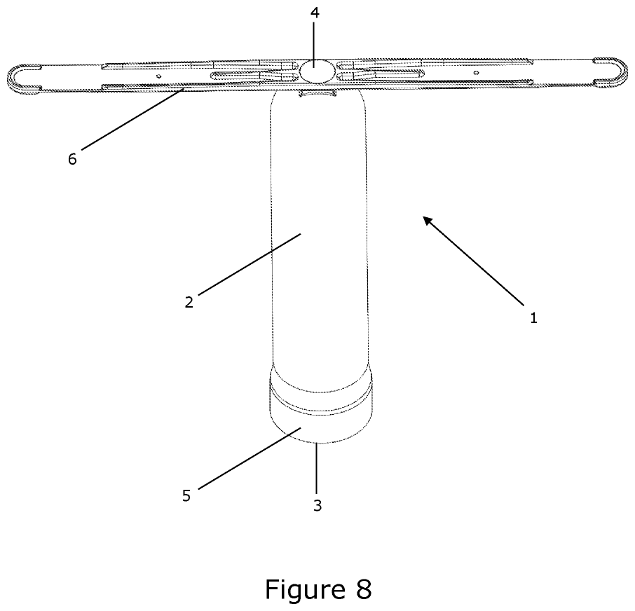

An intra-ruminal device 1 is shown in FIG. 8. The body 2 of the intra-ruminal device is formed of a material that is substantially impervious to rumen fluids. So called “Laby devices” (named after the inventor Ralph Laby), operate on the basis that the medicament in the barrel is exposed to the ruminal fluid at the orifice, or aperture end of the device only. As the medicaments typically swell upon exposure to ruminal fluid, swelling of the medicament in the barrel at locations other than at the orifice or aperture end may alter the operation of the device.

The body 2 of the intra-ruminal device defines a barrel having a first end 3 and a second end 4, and an opening at the first end 3, covered by a cap 5 with multiple holes. The second end 4 is typically sealed.

A Laby device is typified by having a biasing mechanism within the barrel that urges the plunger towards the first end, maintaining the medicament in the barrel at the orifice or aperture at the first end.

Preferably the intra-ruminal device is adapted for continuous or sequential release of the active agent or agents via the cap aperture, reliant on an internal core or column of matrices (i.e. tablets), at least some of which contain the active agent or agents. The active agent or agents are released in to the animal in a controlled manner by contact of matrices with intra-ruminal fluid via the cap orifice or aperture thereby allowing erosion or dissolution of the matrices into the animal.

The biasing arrangement is located within the body distal to the active agents, and involves some form of biasing mechanism such as a spring and plunger, to urge the active agent or agents to the outlet end. Other mechanisms may be used such as the use of gas to create pressure on the plunger or electrical mechanical means.

The intra-ruminal device includes a variable geometry device 6 to ensure retention in the rumen. Ruminants typically regurgitate their food as part of the digestive process, and without a retention mechanism the device 1 can be ejected from the animal.

The body 2 of the intra-ruminal device 1 may be formed into a number of suitable shapes that are able to be administered via the animal's esophagus. Preferably the body 2 of the intra-ruminal device 1 is cylindrically shaped, and preferably the cross section of the body is circular.

The at least one outlet may be located at one end of the body. The outlet may be from about 1, 2, 3, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22 mm or more in diameter, and useful ranges may be selected in between any of these values (for example from about 1-22, in diameter). It will be understood by a person skilled in the art that the size of the outlets will depend on factors such as for example, the intended payout rate.

One or both ends of the body may taper in to a reduced diameter to aid the passage of the intra-ruminal device down the oesophagus to the rumen.

The diameter of the body of the intra-ruminal device is small enough to pass down the oesophagus of a ruminant animal with ease and large enough to accommodate one or more matrices in the barrel. The diameter of the barrel depends on, for example the thickness of the body of the intra-ruminal device. In some embodiments the diameter of the intra-ruminal device 1 and the diameter of the barrel may be very similar, the difference being the result of the thickness of the body.

The diameter of the intra-ruminal device may be less than about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9 or 4 cm, and useful ranges may be selected from any of these values (for example the diameter of the intra-ruminal device may be from about 1 to about 4 cm, or from about 1.2 to about 3.5 cm).

The length of the body of the device can vary to, for example, accommodate more or less matrices. The length of the body may also vary depending on, for example, the animal to which the intra-ruminal device will be administered the size of the animal, the dose and pay-out period and may be from about 70, 75, 80, 85, 90, 95, 100, 105, 110, 115, 120, 125, 130, 135, 140, 145, 150, 155, 160, 165, 170 mm or more, and useful ranges may be selected from any of these values (for example from about 70 mm to about 170 mm, or from about 75 mm to about 165 mm). For example in some embodiments the length of the body of an intra-ruminal device to be administered to sheep and other small ruminants may be from about 76 mm to about 83 mm, and the length of the body of the intra-ruminal device to be administered to cattle and other similar-sized ruminants may be from about 97 to about 162 mm.

### 1.1 Biasing Arrangement

The intra-ruminal device comprises a biasing arrangement located in the barrel of the device within the body, and is adapted to bias the active agent or agents in the barrel to the outlet at the first end of the barrel. In some embodiments, the biasing arrangement may comprise a plunger and biasing means, the plunger defining a space within the barrel between the plunger and the closed end of the barrel. In various embodiments, the biasing means may comprise one or more springs, gas inflation, or electrical mechanical means.

The biasing means, such as a spring, may be made of materials such as alloys of steel, for example stainless steel, carbon steel, oil tempered wire, chrome silicon steel or chrome vanadium steel. Other alloys may also be used, for example Inconel, Monel, beryllium, copper or phosphor bronze. Other suitable materials will be apparent to those skilled in the art.

The biasing arrangement may be adapted to be extendible to at least about 45, 50, 55, 60, 65, 70, 75, 80, 85, 90, 95 or 100% of the length of the body, and suitable ranges may be selected from any of these values (for example from about 45% to about 100%, or from about 80% to about 100%).

The biasing arrangement may comprise a spring that is adapted to push a plunger to extend the biasing arrangement to at least about 80, 85, 90, 95 or 100% of the length of the body.

Without wishing to be bound by theory the inventors believe the pressure exerted by the biasing arrangement, that is, the pressure biasing the active agent or agents towards the at least one outlet contributes to control of the payout period.

The pressure exerted by the compression arrangement, for example the biasing means such as a spring, may remain substantially constant for the entire payout period.

A number of factors can affect the function of the compression arrangement, and these must be tightly controlled to achieve constant, consistent and reliable payout of the one or more active ingredients in the barrel. For example, in conventional devices the permeability of gasses may be compromised, leading to the formation of a partial vacuum in the space between part of the biasing arrangement, for example the plunger of the biasing arrangement, and the closed end of the barrel. This can then lead to inconsistent payout.

The biasing arrangement comprises a plunger and a biasing means, for example one or more springs.

The plunger contacts the inner wall surface of the barrel to substantially form a seal within the barrel.

### 1.2 Variable Geometry Retention Means

The retention means may comprise a variable geometry device, preferably a retractable resilient wing or wings, preferably on one end of the body. The retention mechanism assists rumen retention by preventing regurgitation of the intra-ruminal device.

The resilient wing may comprise an extended position and a retracted position.

The resilient wing may be in an extended position when no force is applied to the wings. In the extended the position the wings extend outwardly from the end of the body distal to the outlet (second end).

The resilient wing may transition from an extended position to a retracted position when a force is applied to a top surface of the wings, such as when the intra-ruminal device 1 is being administered into an internal cavity of an animal. As a force is applied to the top surface of the wings, the variable geometry device, preferably the wings are pressed against the side of the body. The intra-ruminal device returns to an extended position after administration to prevent regurgitation. In some embodiments the intra-ruminal device may comprise more than one retention means, for example a variable geometry device such as a wing or pair of wings and a weighted component.

The variable geometry device, for example wings may be pressed against the side of the body using a pharmaceutical grade polymer or co-polymer tape that is readily dissolved by the contents of the rumen or using a polymer or co-polymer that melts at the temperature of the rumen, for example a polymer that melts at a temperature of from about 37.5, 38, 39, 39.5, 40, 40.5 or 41° C., and useful ranges may be selected from any of these values (for example from about 39 to about 40° C., or from about 38 to about 41° C.). Preferably the melting point of the polymer or co-polymer is from about 38.5 to about 40.5° C. to avoid the polymer melting in the oesophagus of the ruminant and releasing the wings from the side of the body before the device enters the rumen.

The variable geometry device, for example wings may be made from the same polymeric material as the body, or they may be made from a different polymeric material. In various embodiments the variable geometry device, for example wings may be made of a polymeric material that is less rigid than the polymer used to make the body, to allow the wings to be retained against the side of the body during administration to an animal. Suitable polymeric materials will be apparent to a person skilled in the art and may include for example any pharmaceutical grade polymers that are sufficiently pliable to be held against the side of the intra-ruminal device when administered. In various embodiments the wings or part of the wings may be made of polypropylene or a co-polymer thereof.

### 1.3 Matrix

The barrel of the intra-ruminal device contains a core of material that may comprise at least one active-containing matrix comprising at least one active ingredient.

The active-containing matrix may be tablets. Preferably the tablets are controlled-release tablets. Preferably at least one of the tablets is an exit dose tablet.

The barrel may comprise a matrix core in the form of a stack of individual tablet.

The at least one matrix may be any shape adapted to fit inside the barrel of the device. In various embodiments, the barrel of the intra-ruminal device may comprise more than one matrix. The form of the matrix may be for example a tablet, a capsule, a caplet or a wafer. In some embodiments, the one or more matrices may be shaped to allow them to align axially with respect to one another along the longitudinal axis of the body of the intra-ruminal device, such that they are sequentially presentable to the rumen, as originally proposed in the Laby device. Preferably the at least one matrix is a tablet, preferably disc-shaped.

The diameter of the one or more matrices may be less than about 9, 10, 11, 12, 13, 14, 15, 16, 17, 18, 19, 20, 21, 22, 23, 24, 25, 26, 27, 28, 29, 30, 31, 32, 33, 34, 35 mm, and useful ranges may be selected from any of these values (for example the diameter of the matrices may be from about 10 to about 35 mm, or from about 11 to about 32 mm). For example in some embodiments one or more matrices for use in intra-ruminal devices to be administered to sheep may be from about 11 to about 15 mm in diameter and one or more matrices for use in intra-ruminal devices to be administered to cows may be from about 15 to about 32 mm in diameter.

The diameter of the one or more matrices comprising the one or more active ingredients must be such that the diameter is small enough to fit into the barrel of the device. For example if the diameter of the barrel of the device is 20 mm, then the matrix may have a diameter of for example around 18 mm.

The device may comprise a plurality of matrices, for example a plurality of tablets, the number of matrices depending on the length of the body of the device and the thickness of the matrix. For example in some embodiments the thickness of the matrices may be from at least about 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75 or 10 mm or more, and useful ranges may be selected between any of these values, for example from about 3 to about 9 mm, or from about 7 to about 9 mm.

The barrel may comprise one matrix only, for example one solid core comprising at least one active ingredient and optionally one or more excipients. In such embodiments the matrix may substantially span the length of the barrel from the biasing arrangement to end of the body comprising the at least one outlet.

The active ingredient(s) may be released in to the rumen in a controlled manner by contact of the composition comprising the active ingredient(s) with the intra-ruminal fluid allowing erosion or dissolution of the composition in to the rumen.

A seal may exist between the rumen-facing end of the matrix comprising the active ingredient(s) and the barrel of the intra-ruminal device. In various embodiments the part of the compression arrangement, for example the plunger, contacts an inner wall surface of the barrel to substantially form a seal within the barrel. Without wishing to be bound by theory the inventors believe that an ineffective seal between the barrel and rumen-facing end of the matrix comprising the active ingredient(s) may allow other surfaces of the matrix or other matrices in the stack to swell, adversely affecting, or stopping reliable payout of the medication.

### 1.4 Active Ingredients

The one or more matrices of the present invention deliver a therapeutic quantity of one or more active ingredients. The active ingredient(s) are delivered from an intra-ruminal device and may be absorbed in to the systemic circulation.

A wide range of active ingredients may be delivered from the at least one matrix in the intra-ruminal devices of the present invention.

The one or more matrices may comprise one or more antibiotics, antifungals, antivirals, steroid hormones, antihistamines, metabolic regulators, productivity regulators, corticosteroids, antiemetics, anti-thyroidal agents, parasiticidal agents, such as for example anthelmintics, nutritional actives or a combination thereof.

The one or more matrices may comprise one or more vitamins, for example vitamin A, vitamin E, vitamin B₁₂, vitamin B₃, d-pantothenic acid (vitamin B₅), folic acid, vitamin B₆, vitamin B₁, vitamin D₃, vitamin C, vitamin B₂. As another example, the nutritional active could be a pro-vitamin, for example beta-carotene or panthenol.

The nutritional active may be an amino acid. Suitable amino acids include but are not limited to the 20 naturally occurring L-amino acids, for example arginine, isoleucine, leucine, lysine, etc.

The nutritional active may be a co-enzyme, for example co-enzyme Q.

The nutritional active may be a mineral. Non-limiting examples of minerals include potassium, sodium, manganese, zinc, iron, calcium, copper, cobalt, iodine, chlorine and selenium. In some embodiments the mineral may be in the form of a suitable salt.

The one or more matrices may comprise one or more anti-microbial ingredients for example antibiotics, antifungals, antivirals, anthelmintics, and the like.

Suitable antibiotic agents may be those that act as inhibitors of cell wall synthesis (e.g. penicillins, cephalosporins, bacitracin and vancomycin), inhibitors of protein synthesis (aminoglycosides, macrolides, lincosamides, streptogramins, chloramphenicol, tetracyclines), inhibitors of membrane function (e.g. polymixin B and colistin), inhibitors of nucleic acid synthesis (e.g. quinolones, metronidazole, and rifampin), or inhibitors of other metabolic processes (e.g. anti-metabolites, sulfonamides, and trimethoprim). Non-limiting examples of antibiotics include polyethers, ionophores such as monensin and salinomycin, beta-lactams such as penicillins, aminopenicillins (e.g., amoxicillin, ampicillin, hetacillin, etc.), penicillinase resistant antibiotics (e.g., cloxacillin, dicloxacillin, methicillin, nafcillin, oxacillin, etc.), extended spectrum antibiotics (e.g., axlocillin, carbenicillin, mezlocillin, piperacillin, ticarcillin, etc.); cephalosporins (e.g., cefadroxil, cefazolin, cephalexin, cephalothin, cephapirin, cephradine, cefaclor, cefamandole, cefmetazole, cefonicid, ceforanide, cefotetan, cefoxitin, cefprozil, cefuroxime, loracarbef, cefixime, cefoperazone, cefotaxime, cefpodoxime, ceftazidime, ceftiofur, ceftizoxime, ceftriaxone, moxalactam, etc.); monobactams such as aztreonam; carbapenems such as imipenem and meropenem; quinolones (e.g., ciprofloxacin, enrofloxacin, difloxacin, orbifloxacin, marbofloxacin, etc.); chloramphenicols (e.g., chloramphenicol, thiamphenicol, florfenicol, etc.); tetracyclines (e.g., chlortetracycline, tetracycline, oxytetracycline, doxycycline, minocycline, etc.); macrolides (e.g., erythromycin, tylosin, tilmicosin, clarithromycin, azithromycin, etc.); lincosamides (e.g., lincomycin, clindamycin, etc.); aminoglycosides (e.g., gentamicin, amikacin, kanamycin, apramycin, tobramycin, neomycin, dihydrostreptomycin, paromomycin, etc.); sulfonamides (e.g., sulfadimethoxine, sulfamethazine, sulfaquinoxaline, sulfamerazine, sulfathiazole, sulfasalazine, sulfadiazine, sulfabromomethazine, suflamethoxypyridazine, etc.); glycopeptides (e.g., vancomycin, teicoplanin, ramoplanin, and decaplanin; and other antibiotics (e.g., rifampin, nitrofuran, virginiamycin, polymyxins, tobramycin, etc.).

The one or more matrices may comprise one or more antifungal active ingredients for example one or more polyenes, azoles, allylamines, morpholines, antimetabolites, and combinations thereof. For example in some embodiments the matrices of the invention may comprise one or more of fluconazole, itraconazole, clotrimazole, ketoconazole, terbinafine, 5-fluorocytosine, and amphotericin B, or combinations thereof.

Non-limiting examples of antivirals that may be present in the one or more matrices of the invention may include didanosine, lamivudine, stavudine, zidovudine, indinavir, and ritonavir.

The one or more matrices may comprise one or more steroid hormone, for example steroid hormones such as growth promoters and production enhancers. In some embodiments, the steroid hormone may be natural steroid hormone, such as for example estradiol, progesterone, and testosterone, or a synthetic steroid hormone, such as trenbolone acetate, estradiol benzoate, estradiol 173, and melengestrol acetate, and/or zeranol.

Steroid hormones that may be present in one or more matrices of the invention may comprise for example natural and synthetic steroid hormones, steroid hormone precursors, steroid hormone metabolites, and derivatives thereof that are structurally derived from cholesterol. Steroid hormones may be synthesized from cholesterol via pathways that involve cytochrome P450 (cP450) enzymes, which are heme-containing proteins.

The one or more matrices may comprise one or more steroid hormones such as for example androgens, estrogens, progestogens, mineral corticoids, and glucocorticoids. Exemplary androgens include, but are not limited to, testosterone, dehydroepiandrosterone, dehydroepiandrosterone sulphate, dihydrotestosterone, androstenedione, androstenediol, androstanedione, androstanediol, and any combination thereof. Exemplary estrogens include, but are not limited to, estrone, estradiol, estriol, estetrol, equilin, equilenin, and any combination thereof. Exemplary progestogens include, but are not limited to, progesterone, 17-hydroxy-progesterone, pregnenolone, dihydroprogesterone, allopregnanolone, 17-hydroxy-pregnenolone, 17-hydroxy-dihydroprogesterone, 17-hydroxy-allopregnanolone, and any combination thereof. Exemplary mineral corticoids include, but are not limited to, aldosterone, 11-deoxycorticosterone, fludrocortisones, 11-deoxy-cortisol, pregnenedione, and any combination thereof. Exemplary glucocorticoids, include, but are not limited to, cortisol (hydrocortisone), corticosterone, 18-hydroxy-corticosterone, cortisone, and any combination thereof.

The one or more matrices may comprise one or more anti-histamines, such as for example clemastine, clemastine fumarate (2(R)-[2-[1-(4-chlorophenyl)-1-phenyl-ethoxy]ethyl-1-methylpyrrolidine), dexmedetomidine, doxylamine, loratadine, desloratadine and promethazine, and diphenhydramine, or pharmaceutically acceptable salts, solvates or esters thereof.

The one or more matrices may comprise one or more metabolic or fermentation regulators, such as for example one or more methane inhibitors or a hypothyroidism treatment.

The one or more matrices may comprise one or more productivity regulators, for example polyethers such as monensin. In some embodiments, the productivity regulator may be a productivity enhancer or feed efficiency enhancer.

The one or more matrices may comprise one or more anthelmintic agents, for example one or more benzimidazoles, imidazothiazoles, tetrahydropyrimidines, macrocyclic lactones, salicylanilides, substituted phenols, aromatic amides, isoquinolines, amino acetonitriles, spiroindoles, or combinations thereof.

Anthelmintic benzimidazoles comprise for example mebendazole, flubendazole, fenbendazole, oxfendazole, oxibendazole, albendazole, albendazole sulfoxide, thiabendazole, thiophanate, febantel, netobimin, and triclabendazole. Further examples include mebendazole, and ricobendazole.

Without wishing to be bound by theory, the inventors believe that benzimidazole-based anthelmintics may interfere with the worm's energy metabolism on a cellular level by binding to a specific building block called beta tubulin and preventing its incorporation into certain cellular structures called microtubules, which are essential for energy metabolism.

Imidazothiazoles and tetrahydropyrimidines are both nicotinic agonists. In some embodiments the one or more anthelmintic agents in the one or more matrices may comprise imidathiazoles, for example levamisole, tetramisole, and butamisole. Tetrahydropyrimidine anthelmintics that may be used in the matrices of the invention include, for example, morantel, oxantel, and pyrantel.

Without wishing to be bound by theory the inventors believe that tetrahydropyrimidines may mimic the activity of acetylcholine, a naturally occurring neurotransmitter that initiates muscular contraction. This may lead to helminths that are unable to feed and starve.

Without wishing to be bound by theory the inventors believe that imidazothiazoles may have a similar mode of action to tetrahydropyrimidines and may cause spastic paralysis of helminths, For example, levamisole is thought to have a broad spectrum of activity and may therefore be effective against many larval stages of parasites.

The one or more matrices may comprise one or more macrocyclic lactones, for example abamectin, doramectin, eprinomectin, ivermectin, selamectin, milbemycin, for example as milbemycin oxime, moxidectin or a combination thereof.

The one or more matrices may comprise one or more salicylanilides for example brotianide, clioxanide, closantel, niclosamide, oxyclozanide, rafoxanide, substituted phenols including for example bithionol, disophenol, hexachlorophene, niclofolan, menichlopholan, nitroxynil, and aromatic amides, including for example diamfenetide (diamphenethide) or combinations thereof.

The one or more matrices may comprise one or more isoquinoline anthelmintics, such as for example praziquantel and epsiprantel. In some embodiments the matrices of the invention and the intra-ruminal devices may comprise one or more amino-acetonitrile derivatives, such as for example monepantel.

The one or more matrices may comprise one or more active ingredients such as for example piperazine and derivatives thereof such as piperazine and diethylcarbamazine (DEC, a derivative of piperazine), benzenesulfonamides such as clorsulon, amidines such as bunamidine, isothiocyantes such as nitroscanate, and organophosphates such as dichlorvos, and spiroindoles such as derquantel (2-deoxoparaherquamide).

The one or more matrices may comprise a ingredient for the control of external parasites such as fleas and ticks or other blood sucking parasitic insects that are active systemically such as isoxazolines

The one or more active ingredient(s) in the at least one matrix of the intra-ruminal device, is/are stable and does not react with other components in the reaction mixture or degrade or decompose by other means.

The payout rates of the active ingredient(s) may be measured as a function of the width of a matrix ejected into the rumen through the one or more outlets in the end cap. In some embodiments the payout rate of the intra-ruminal device of the invention may be from about 0.1, 0.125, 0.15, 0.175, 0.2, 0.225, 0.025, 0.275, 0.3, 0.325, 0.35, 0.375, 0.4, 0.425, 0.45, 0.475, 0.500, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95, 0.975, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9 to 3 mm or more per day in water, and suitable ranges may be selected from any of these values, for example from about 0.3 mm to about 2.4 mm, or from about 0.5 mm to about 1.5 mm). Preferably, the payout of the one or more active ingredients is linear and in various embodiments the linearity may be greater than 0.95.

The payout rates of the one or more active ingredient(s) may be minimally affected, preferably not affected by the pH and ionic composition of the rumen.

The one or more matrices of the intra-ruminal device may comprise more than one active ingredient. For example in some embodiments the matrices of the invention may comprise from 2, 3, 4, 5, 7, 8, 9, or about 10, or more active ingredients, and useful ranges may be selected from any of these values (for example from 2 to about 10 or from 2 to about 5 active ingredients).

The one or more matrices of the intra-ruminal device may comprise more than one active ingredient, wherein some or all of the active ingredients belong to a different therapeutic class, for example antibiotics, antifungals, antivirals, steroid hormones, antihistamines, metabolic regulators, productivity regulators, corticosteroids, antiemetics, anti-thyroidal agents, parasiticidal agents, such as for example anthelmintics and/or nutritional actives. For example the matrix may comprise 3 actives, one of which is an anthelmintic, one of which is an antibiotic and the third one may be a nutritional active, for example a vitamin.

The one or more matrices of the intra-ruminal device may comprise more than one active ingredient, each of which belongs in the same therapeutic class, preferably anthelmintics. In some embodiments the matrix may comprise two or more anthelmintic actives belonging to the same class of anthelmintics, such as for example benzimidazoles, imidazothiazoles, tetrahydropyrimidines, macrocyclic lactones, salicylanilides, substituted phenols, aromatic amides, isoquinolines, amino acetonitriles and spiroindoles. For example the matrices may comprise two or three actives, each of which may be a macrocyclic lactone.

The one or more matrices of the intra-ruminal device may comprise two or more active ingredients each of which is an anthelmintic active and each belonging to a different anthelmintic class, such as for example benzimidazoles, imidazothiazoles, tetrahydropyrimidines, macrocyclic lactones, salicylanilides, substituted phenols, aromatic amides, isoquinolines, amino acetonitriles and spiroindoles. For example the matrices may comprise two anthelmintics, one of which may be a macrocyclic lactone and the other may be an imidazothiazole.

The one or more matrices may comprise at least about 5, 7.5, 10, 12.5, 15, 17.5, 20, 22.5, 25, 27.5, 30, 32.5, 35, 37.5, 40, 42.5, 45, 47.5, 50, 52.5 or 550/o or more of one or more active ingredients by weight of each matrix, and useful ranges may be selected from any of these values (for example from about 8 to about 50% or from about 10 to about 40% by weight of the matrix).

### 1.5 Other Ingredients

The one or more matrices comprising the one or more active ingredients and polymers may further comprise a number of excipients. Examples of suitable excipient may include, but are not limited to fillers, diluents, lubricants, surfactants, glidants, gel formers, binders, and stabilisers, or combinations thereof.

The one or more matrices of the invention may further comprise one or more fillers or diluents. Examples of suitable fillers or diluents may include, but are not limited to, sugars such as for example lactose, sucrose and mannitol, inorganic salts such as calcium phosphate and calcium carbonate, cellulose, methyl cellulose, ethyl cellulose, aluminium silicates such as kaolin or combinations thereof.

The one or more matrices may comprise one or more fillers and/or diluents at amounts of from about 0, 0.1, 0.25, 0.5, 0.75, 1, 2, 3, 4, 5, 6, 7, 8, 9, 10, 12.5, 15, 17.5, 20, 22.5, 25, 27.5, 30, 32.5, 35, 37.5, 40, 42.5, 45, 47.5, 50, 52.5, 55, 57.5, 60, 62.5, 65, 67.5, 70, 72.5, 75, 77.5, 80, 82.5, 85, 87.5, 90, 92.5, 95% by weight of the matrix, and useful ranges may be selected from any of these values (for example from about 0.1 to about 80% by weight or from about 0.1 to about 15% by weight of the matrix).

The one or more matrices may comprise one or more surfactants or lubricants. Examples of surfactants or lubricants may include, but are not limited to, metal stearates such as for example metal or non-metal stearates such as magnesium stearate, calcium stearate and stearyl fumarate, glyceryl stearates such as for example glyceryl monostearate, glycerine derivatives, sodium lauryl sulfate, sucrose fatty acid ester, mineral clays, and aluminium silicates such as kaolin or combinations thereof.

One or more surfactants and/or lubricants may be present in the matrices of the invention in an amount of from about 0, 0.01, 0.05, 0.075, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, or 90% by weight of the matrix, and useful ranges may be selected from any of these values (for example from about 0.5 to about 50% or from about 5% to about 40%).

The one or more matrices may further comprise one or more glidants. Examples of glidants include, but are not limited to, colloidal silica dioxide, talc, metal stearates such as magnesium stearate, calcium stearate and stearyl fumarate, and glyceryl stearates such as glyceryl monostearate, or combinations thereof

The glidant(s) may be present in the one or more matrices in amounts of from about 0, 0.25, 0.5, 0.75, 1, 1.25, 1.5, 1.75, 2, 2.25, 2.5, 2.75, 3, 3.25, 3.5, 3.75, 4, 4.25, 4.5, 4.75, or 5% by weight of the matrix, and useful ranges may be selected from any of these values (for example from about 0.25 to about 4% or from about 0 to about 2% by weight of the matrix).

The one or more matrices may comprise one or more additional gel formers. Examples of additional gel formers that may be used include, but are not limited to, sucrose fatty acid ester, cellulosic derivatives such as hydroxyethyl cellulose and hydroxymethyl cellulose, chitosan, polyethylene oxide, carbopol or combinations thereof

The gel former(s) may be present in the one or more matrices in amounts of from about 0, 0.1, 0.25, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, 50, 55, 60, 65, 70, 75, 80, 85 or 90% by weight of the matrix, and useful ranges may be selected from any of these values (for example from about 0.5 to about 80% or from about 1% to about 50% by weight of the matrix).

The one or more matrices may comprise one or more binders. Examples of binders include, but are not limited to, cellulosic derivatives such as hydroxyethyl cellulose and hydroxymethyl cellulose.

The binder(s) may be present in the one or more matrices of the invention in amounts of from about 0, 0.1, 0.25, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, or 50% by weight of the matrix, and useful ranges may be selected from any of these values (for example From about 0 to about 35% by weight of the matrix.

The one or more matrices may comprise one or more stabilisers. Examples of stabilisers that may be used in the matrices include, but are not limited to, antioxidants such as for example butylated hydroxytoluene, butylated hydroxyanisole and tocopherol, and buffers.

The stabilisers(s) may be present in the one or more matrices in amounts of from about 0.01, 0.02, 0.03, 0.04, 0.05, 0.06, 0.07, 0.08, 0.09, 0.1, 0.15, 0.2, 0.25, 0.5, 0.75, 1, 1.25, 1.5, 1.75, 2, 2.25, 2.5, 2.75, 3, 3.25, 3.5, 3.75, 4, 4.25, 4.5, 4.75, or 5% by weight of the matrix, and useful ranges may be selected from any of these values (for example from about 0.1 to about 5% or from about 0.5 to about 3.5% by weight of the matrix).

### 2. Intra-Ruminal Device Cap

The intraluminal device comprises a cap that seals the first end of the device to retain the matrix of active agents within the barrel. The cap encapsulates the end of the barrel, and contains at least two or more apertures.

The cap outlet may comprise 2, 3, 4, 5, 6, 7, 8, 9 or 10 apertures, and suitable ranges may be selected from between any of these values, (for example, about 2 to about 10, about 2 to about 8, about 2 to about 7, about 2 to about 6, about 2 to about 5, about 2 to about 4, about 2 to about 3, about 3 to about 10, about 3 to about 9, about 3 to about 8, about 3 to about 6, about 3 to about 5, about 4 to about 10, about 4 to about 8, about 4 to about 7, about 5 to about 10, about 5 to about 9, about 5 to about 7, about 6 to about 10 or about 6 to about 9).

The apertures may comprise two or more symmetrical shapes distributed equally around the orifice cap to ensure uniform hydration of the matrix at the cap end exposed to the rumen fluids

The diameter of an aperture may be at least about 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8 or 5.0 mm, and suitable ranges may be selected from between any of these values (for example, about 1.0 to about 5.0, about 2.0 to about 5.0, about 2.0 to about 4.4, about 2.0 to about 4.0, about 2.0 to about 3.6, about 2.0 to about 3.2, about 2.2 to about 5.0, about 2.2 to about 4.6, about 2.2 to about 4.0, about 2.4 to about 5.0, about 2.4 to about 4.6, about 2.4 to about 3.8, about 2.4 to about 5.0, about 2.4 to about 4.8, about 2.4 to about 4.2, about 2.6 to about 5.0, about 2.6 to about 4.4, about 2.6 to about 4.0, about 2.8 to about 5.0, about 2.8 to about 4.4, about 3.0 to about 5.0, about 3.0 to about 4.6, about 3.0 to about 4.2, about 3.2 to about 5.0, about 3.2 to about 4.8, about 3.2 to about 4.2, about 3.4 to about 5.0, about 3.4 to about 4.6, about 3.4 to about 4.2, about 3.6 to about 5.0, about 3.6 to about 4.6, about 3.6 to about 4.0, about 3.8 to about 5.0, about 3.8 to about 4.8 or about 4.0 to about 5.0 mm).

Preferably the total surface area of the apertures of the multiple-aperture cap of the present invention is within 1, 5, 10, 15, 20, 25, 30, 35 or 40% of a typical single aperture cap, and suitable ranges may be selected from between any of these values (for example, about 1 to about 40, about 1 to about 35, about 1 to about 30, about 1 to about 25, about 1 to about 20, about 1 to about 15, about 1 to about 10, about 1 to about 5, about 5 to about 40, about 5 to about 30, about 5 to about 20, about 5 to about 10, about 10 to about 40, about 10 to about 35, about 10 to about 30, about 10 to about 20, about 15 to about 40, about 15 to about 30, about 15 to about 25, about 20 to about 40, about 20 to about 35, about 20 to about 30, about 25 to about 40, about 25 to about 35 or about 30 to about 40%).

The total surface area of the apertures of the multiple-aperture cap of the present invention is about 10, 15, 20, 25, 30, 35, 40, 45, 50, 55, or 60 mm², and suitable ranges may be selected from between any of these values, (for example, about 10 to about 60, about 10 to about 50, about 10 to about 40, about 15 to about 60, about 15 to about 50, about 15 to about 45, about 15 to about 30, about 20 to about 60, about 20 to about 55, about 20 to about 45, about 25 to about 60, about 25 to about 50, about 25 to about 40, about 30 to about 60, about 30 to about 5, about 35 to about 60, about 35 to about 55 or about 40 to about 60 mm²).

The cap may be ultrasonically welded to the barrel.

The apertures of the multiple-apertured caps may have the same diameter. The apertures may be shaped as a circle, oval, square, squircle or rectangle.

The apertures of the multiple-apertured caps have a diameter that is within 2, 4, 6, 8, 10, 12, 14, 16, 18 or 20% of their average size, and suitable ranges may be selected from between any of these values, (for example, about 2 to about 20, about 2 to about 18, about 2 to about 14, about 2 to about 12, about 2 to about 10, about 4 to about 20, about 4 to about 18, about 4 to about 12, about 4 to about 10, about 6 to about 20, about 6 to about 18, about 6 to about 12, about 6 to about 10, about 8 to about 20, about 8 to about 16, about 8 to about 12, about 8 to about 10, about 10 to about 20, about 10 to about 16, about 12 to about 20%).

The body and the cap are held together under pressure, or heat and pressure.

The cap encapsulates the first end outlet of the device, the cap comprising two or more apertures, and in some embodiments at least one variable geometry device dependent from the body to assist rumen retention, or both.

The cap may be made of the same material as the body or a different material. In various embodiments the cap is made of a polymeric material that is stable under the conditions present in the rumen of the animal. For example, the end cap may be made of polypropylene or a co-polymer of polypropylene.

### 3. Method of Assembling Intra-Ruminal Device

The method of assembling an intra-ruminal device as described above includes first loading the active agent into the barrel. The cap is then located over the opening at the first end.

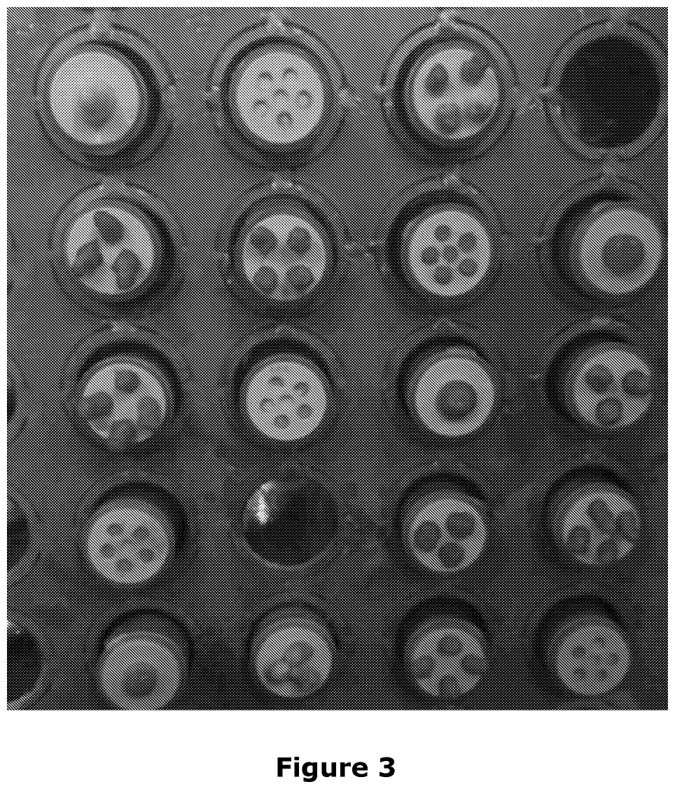

To assemble the intra-ruminal device 1, the device transforms from a pre-weld condition as shown in FIG. 3, to an intermediate condition as the protrusions begin to melt, to a fixed condition where the protrusions have melted and the base of the cap 19 is push up against the end of the barrel 20.

The cap and barrel may include a snap fit protrusion shown as item 5 (and the corresponding thickening of the terminal portion of the cap 21) in FIG. 3. That is, the cap is pushed onto the end of the barrel until the terminal thickening of the cap 21 snaps over the protrusion 5. This enables the cap to be held loosely on the end of the barrel awaiting welding and fitting.

In one configuration the welded capsules are for use in cows.

### 4. Use of the Capsule

The capsules may have a pay-out (release rate) linearity of at least 0.940, 0.945, 0.950, 0.955, 0.960, 0.965, 0.970, 0.975, 0.980, 0.985, 0.990 or 0.995, and suitable ranges may be selected from between any of these values.

The linearity may be measured over at least 80, 85, 90, 95, 100, 105, 110, 115 or 120 days, and suitable ranges may be selected from between any of these values.

The capsules may have pay-out (release rate) of about 0.90, 0.95, 1.00, 1.05, 1.10, 1.15 or 1.20 mm/day, and suitable ranges may be selected from between any of these values.

The intra-ruminal device may be used in a group or herd of ruminants, each ruminant being administered an intra-ruminal device, and wherein the variable retention means remain attached to the body for the duration of the active agent payout period.

The variable geometry retention means may be designed to separate from the body after the payout period.

At least 80, 81, 82, 83, 84, 85, 86, 87, 88, 89, 90, 91, 92, 93, 94 or 95% of each capsule may extrude before the next capsule in the stack of capsule begins to extrude, and suitable ranges may be selected from between any of these values.

Adjacent capsules in the stack of capsules may exhibit a reduction in co-extrusion.

### EXAMPLES

### 1. Example 1—Manufacture and Evaluation of Exit-Dose Sheep Capsules

### 1.1 Purpose

The purpose of this study was to manufacture and evaluate controlled-release capsules containing an exit-dose tablet. The capsules were assembled using multi-hole orifices and their performance evaluated using an in vitro testing tank.

### 1.2 Methodology

Two batches of tablets were manufactured using a Turbula T2 Mixer and compressed using a single station press fitted with 14.5 mm flat bevelled-edge plain tooling. A first batch had a batch size of 500 g and contained an exit dose of levamisole and oxfendazole with a red dye (Exalake red dye). The second batch had a batch size of 1000 g and was a controlled-release placebo formulation.

Capsules were assembled using five placebo tablets in front of a single exit-dose tablet. Four orifice designs were evaluated using the in vitro Tank tester.

Each of the exit dose tablets weighed between 0.961 and 1.011 g, with a target weight of 1.00 g. Furthermore, each tablet had a thickness of between 5.11 and 5.25 mm, with a target thickness of 5.2 mm. Lastly, each tablet had a hardness of 11.2 to 14.8 kP.

Each of the placebo dose tablets weighed between 0.974 and 0.998 g, with a target weight of 1.00 g. Furthermore, each tablet had a thickness of between 5.26 and 5.33 mm, with a target thickness of 5.2 mm. Lastly, each tablet had a hardness of 8 to 9 kP.

Four different capsule assemblies were tested as follows:

a) Traditional orifice design as a control.

b) Multi-hole orifice design (3 holes)

c) Multi-hole orifice design (4 holes)

d) Multi-hole orifice design (6 holes)

The capsule design is described in Table 1 below.

TABLE 1
Capsule design

| Test group | Orifice design | Orifice total surface area |
|---|---|---|
| A | 1 × 7 mm | 38.5 |
| B | 3 × 4 mm | 37.7 |
| C | 4 × 3.5 mm | 38.5 |
| D | 6 × 3 mm | 42.4 |

Once assembled, the capsules were placed into a tank plate in a testing tank. As the capsule was extruded out of the orifice, a wiper brush mechanism was used to remove extrudate from the capsule.

### Results

The results are summarised in Table 2 summarises the in vitro pay-out data for the four trials. Table 2 shows that as the number of holes in orifice plate increased, there was an elevation in pay-out rate. It should be noted, that the total surface area of capsules from design group (d) was approximately 10% higher than that of the other trials which may account for some of the increase observed for this trial.

TABLE 2
Exit dose kinetic data

| Test group | Mean Pay-out (mm/day) | Minimum R² | % CV |
|---|---|---|---|
| A | 1.193 | 0.935 | 3.92 |
| B | 1.295 | 0.961 | 3.11 |
| C | 1.450 | 0.982 | 6.07 |
| D | 1.737 | 0.980 | 3.58 |

All four trials were run simultaneously in the same tank so any variability in wiping does not affect the comparison between trials.

By day 14 of the trial, capsules from test group (d) was almost complete with the red dye (corresponding to the exit dose tablet) clearly visible for all six capsules at the orifice apertures. Importantly, the dye was consistently observed across the orifices indicating that the exit dose had minimal co-extrusion with the tablet in front. Indeed, the red dye was also observed across all four orifices for one capsule from test group (c). White tablets were clearly visible for capsules from test group (a) and test group (b) indicating that they were running slower than the other trials.

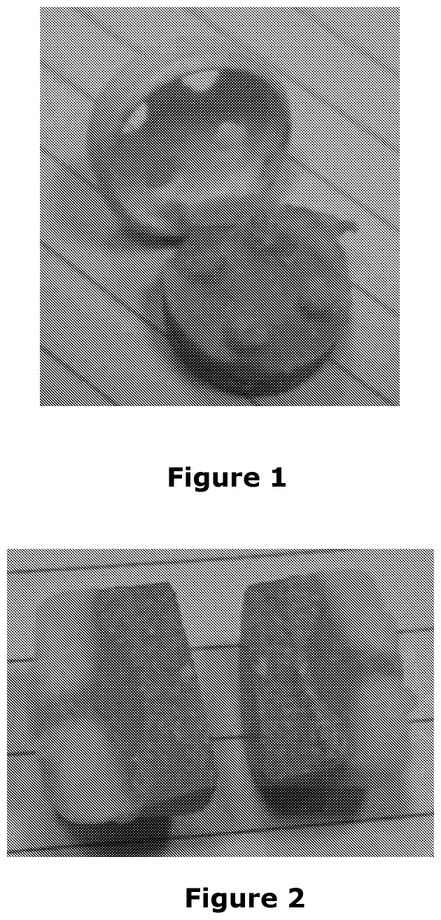

Similar observations were also made on day 17. As shown in FIG. 1, when a capsule from test group (c) was opened up, only a very thin layer of placebo tablet was observed in front of the near full height exit-dose tablet. This indicates that minimal co-extrusion takes place with the 4-hole orifice.

FIG. 2 shows a digital photographs of a capsule from test group (a) (single orifice control). This capsule was opened up as soon as red dye was observed at the centre of the orifice aperture. The core was then cross-sectioned down through the middle of the dye using a surgical scalpel. FIG. 2 shows a ‘volcano’ effect whereby wetting of the exit-dose tablets has resulted in significant amounts of co-extrusion, of almost a tablet thickness, with the tablet in front.

On day 18 the capsules were lifted out of the water and out of contact with the wiper brush but kept in the heated humid tank environment. When observed three days later the devices had continued to extrude. However, as the wiper brush was turned off, the gel had not been removed (see FIG. 3). Most of the capsules were near completion and consequently intense red extrusions of the exit tablet was observed for most of the remaining capsules. The extrudate from the slowest single aperture capsule positioned in the top left (indicted by yellow circle) was observed to be pale pink, indicating the exit-dose tablet was in the early stages of co-extruding with the tablet in front.

Increasing the number of holes in the orifice plate was shown to increase the pay-rates determined in vitro. When a conventional single-hole orifice was employed, significant co-extrusion of the exit-dose tablet was observed to occur with the tablet in front. Importantly, this effect was markedly reduced when multi-hole orifices were used.

### 2. Example 2—In Vitro and In Vivo Evaluation of Pulse and Exit-Dose Sheep Capsules

### 2.1 Purpose

The purpose of this study was to examine the manufacture and evaluation of capsule exit-dose and pulse-release capsules. Capsules were assembled into exit-dose and pulse-release arrangements using standard single and novel multi-hole orifices and their performance evaluated in vitro using a model tank and in vivo using fistulated cattle.

### 2.2 Methodology

This work was split into three studies. Study A assessed the in vivo performance of an exit-dose capsule which was previously evaluated in vitro in Example 1. Studies B and C investigated the performance of pulse-release capsules in vitro and in vivo, respectively.

### Study A: Influence of Orifice Design on In Vivo Performance of an Exit-Dose Capsule

Study A examined the influence of orifice design on the in vivo pay-out of controlled-release capsules containing exit-dose tablets. To perform this study, two types of tablet and three types of orifice were used:
Controlled-release placebo tablets (CR): CR tablets were manufactured as per Example 1 using placebo granules from production.
Exit-dose tablets (E): The ‘exit’-dose formulation containing oxfendazole and levamisole hydrochloride was employed as the exit-dose tablet. The tablets also contained a red dye so they could be easily visualised during extrusion.
Standard Orifice Design: Conventional 7 mm orifices were used.
Multi-hole orifices: Two novel orifice plate designs were machined in order to create 4 or 6 small holes with a combined surface area equivalent to the standard 7 mm orifice (approximately 38 mm² of aperture area).

Kinetic performance was evaluated in 14 to 16 year old rumen-fistulated ex-dairy cows.

The capsule design is described in Table 3 below.

TABLE 3
Study A capsule design

| | | |
|---|---|---|
| Test group | Orifice design | Orifice total surface area |

| | | | |
|---|---|---|---|
| E | 1 × 7 | mm | 38.5 |
| F | 4 × 3.5 | mm | 38.5 |
| G | 6 × 2.87 | mm | 38.8 |

Study B: Influence of Orifice Design on the In Vitro Performance of a Pulsatile-Dose Capsule

Table 2 shows that Study B evaluated positioning the ‘exit’-dose tablet at mid- and end-core (test group H, I, J) and at the start, middle and end of the core (test group K, L and M). Three orifice designs were studied for each tablet configuration.

TABLE 4
Study B capsule design

| Test group | Orifice design | | Tablet arrangement |
|---|---|---|---|
| H | 1 × 7 | mm | 3CR, 1E, 3CR, 1E |
| I | 4 × 3.5 | mm | |
| J | 6 × 2.87 | mm | |
| K | 1 × 7 | mm | 1E, 3CR, 1E, 3CR, 1E |
| L | 4 × 3.5 | mm | |
| M | 6 × 2.87 | mm | |

Study C: Influence of orifice design on the in vivo performance of a pulsatile-dose capsule

Study C evaluated positioning the ‘exit’-dose tablet mid and end-core (test group N and O) and at the start, middle and end of the core (test group P and Q). Two orifice designs were studied for each tablet configuration (Table 3).

TABLE 5
Study C capsule design

| Test group | Orifice design | | Tablet arrangement |
|---|---|---|---|
| N | 1 × 7 | mm | 3CR, 1E, 3CR, 1E |
| O | 6 × 2.87 | mm | |
| P | 1 × 7 | mm | 1E, 3CR, 1E, 3CR, 1E |
| Q | 6 × 2.87 | mm | |
| R | 1 × 7 | mm | 3CR, 1E, 3CR, 1E |
| S | 6 × 2.87 | mm | |
| T | 1 × 7 | mm | 1E, 3CR, 1E, 3CR, 1E |
| U | 6 × 2.87 | mm | |

### 2.3 Results

### Study A: Influence of Orifice Design on In Vivo Performance of an Exit-Dose Capsule

Table 6 summarises the pay-out results from the three in vivo trials and the profiles are illustrated in FIG. 1. It can be seen that the pay-out rate was higher when the number of holes in the orifice plate was increased. This is in agreement with in vitro results reported in Example 1. Linearity results were >0.99 and the % CV was less than 5% for all three trials. The shaded area in FIG. 1 illustrates the pay-out region where the exit-dose tablet is being extruded. It can be seen that the pay-out rate of the exit-dose tablet appears similar to the CR tablets.

TABLE 6
Pay-out summary results for trials with
capsules from Test Groups E, F and G

| Test group | Mean Pay-out (mm/day) | Minimum R² | % CV |
|---|---|---|---|
| E | 1.000 | 0.993 | 3.78 |
| F | 1.276 | 0.991 | 3.10 |
| G | 1.293 | 0.997 | 3.65 |

Multi-hole capsules were removed from the rumen at day 21. The capsules were opened up and the lead tablet cross-sectioned. At day 21 no placebo tablet remained. A single-hole capsule was removed as soon as dye was observed at the orifice (day 24). A large proportion of the placebo tablet in front of the exit tablet was yet still to extrude and exhibited a ‘volcano’ appearance consistent with what was observed in Example 1.

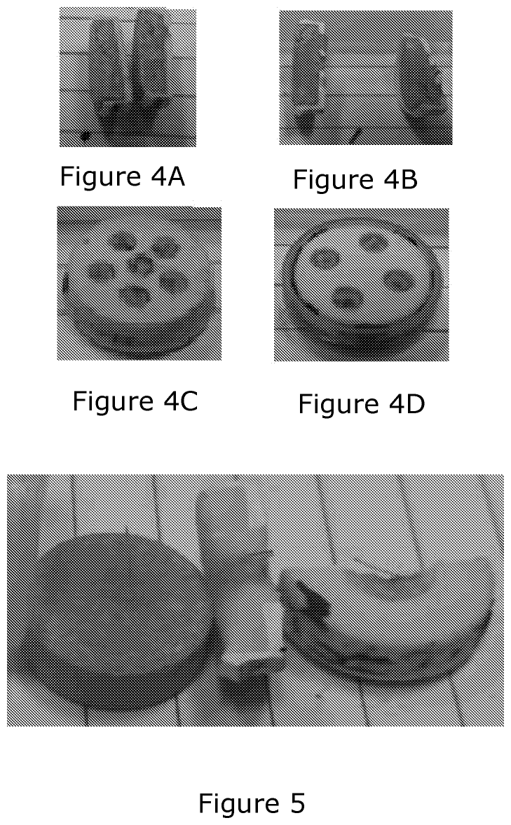

FIG. 4 shows examples of multi-hole capsules removed from the rumen at day 21. The capsules were opened up and the lead tablet cross-sectioned. It can be seen that at day 21 no placebo tablet remained.

FIG. 5 shows a single-hole capsule which was removed as soon as dye was observed at the orifice (day 24). It can be seen that quite a large proportion of the placebo tablet in front of the exit tablet was still to extrude. This classical ‘volcano’ appearance is consistent with what was observed in vitro (see Example 1).

### Study B: Influence of Orifice Design on the In Vitro Performance of a Pulsatile-Dose Capsule

Table 5 summarises the pay-out results from the in vitro trials performed using the pulsatile-dose device. It should be noted that trials L and M were stopped after only 3 days and therefore no results are reported. Both of these trials had a ‘pulse-release’ tablet as the first tablet in the capsule and also had multiple holes in the orifice. Significant over-wetting of the tablet core was observed in these trials with red dye observed throughout the capsule (FIG. 4). Therefore, it was decided to stop these two trials.

TABLE 7
Pay-out summary results for trials with capsules
from Test Groups H, I, J, K, L and M

| Test group | Mean Pay-out (mm/day) | Minimum R² | % CV |
|---|---|---|---|
| H | 0.946 | 0.92 | 10.3 |
| I | 1.078 | 0.94 | 13.5 |
| J | 0.948 | 0.84 | 15.8 |
| K | 0.993 | 0.94 | 22.6 |

| | |
|---|---|
| L | no results obtained |
| M | no results obtained |

Table 7 illustrates the pay-out profiles of the four trials which were ran out to day 47 in vitro. Trials with capsules from test groups H, I and J were configured with pulse-release tablets only in the middle and end positions of the tablet core. All three trials started up as expected and ran linear until around day 14 (equivalent to approximately 16 mm core travel). Each placebo tablet was approximately 5.3 mm in thickness. Therefore, 16 mm corresponds to the location of the first pulse-dose tablet in these trials. It can be seen that at this point, the capsules essentially stalled for a number of days during which over-wetting of the core was observed. The over-wetting is likely to be the reason for the stalling.

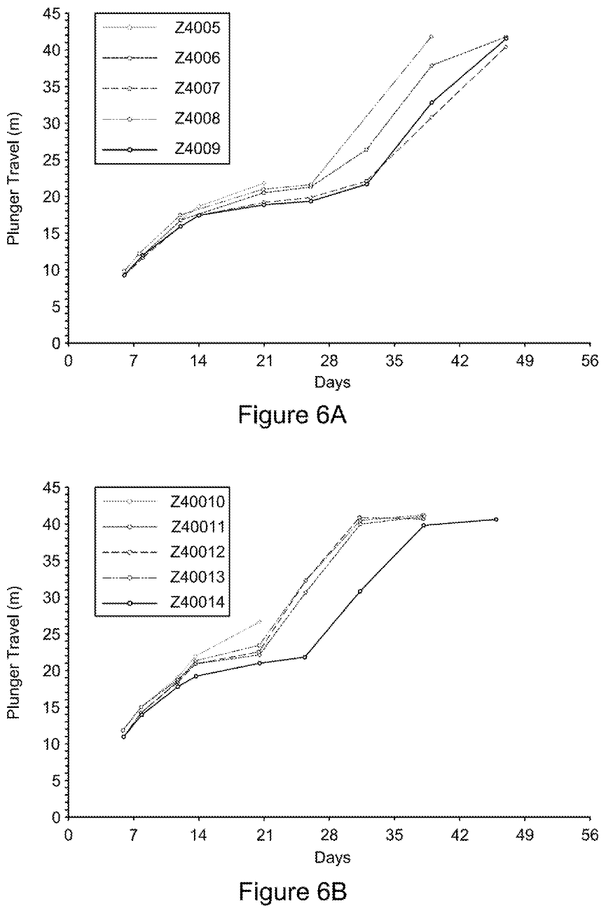

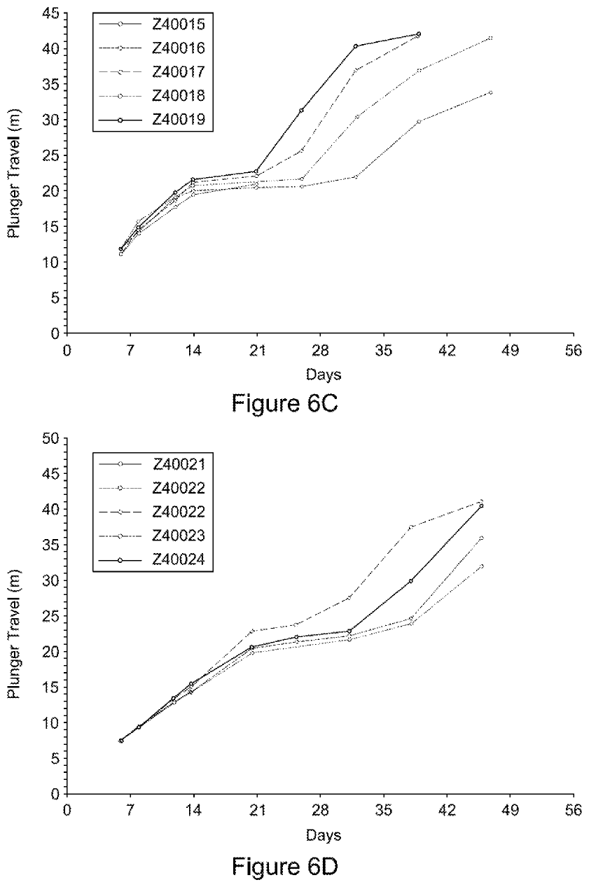

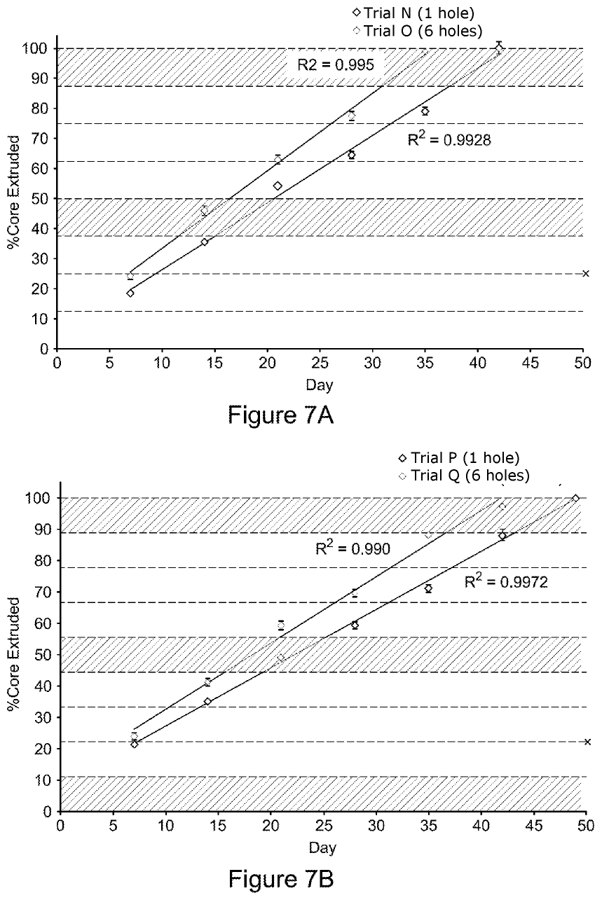

FIG. 6 illustrates the pay-out profiles of trials H, I, J and K which were run out to day 47 in vitro. Trials H and J were configured with pulse-release tablets only in the middle and end positions of the tablet core. All three trials started up as expected and ran linear until around day 14 (equivalent to approximately 16 mm core travel). Each placebo tablet was approximately 5.3 mm in thickness. Therefore, 16 mm corresponds to the location of the first pulse-dose tablet in these trials. It can be seen that at this point, the capsules essentially stalled for a number of days during which over-wetting of the core was observed. The over-wetting is likely to be the reason for the stalling.

Trial with the capsule from Test Group K has a pulse-release tablet as the first tablet in the core and used a single-hole orifice. No marked over-wetting or stalling was observed at start-up. However, the capsules did stall at approximately day 21 (22-23 mm of plunger travel) which correlates to the pulse-tablet in the middle of the capsule device. The capsules did re-start but at varying pay-out rates.

In summary, this work showed that when placed at the start or middle of the core, the ‘pulse’ tablet has the potential to rapidly wet which can lead to stalling in vitro.

### Study C: Influence of Orifice Design on the In Vivo Performance of a Pulsatile-Dose Capsule

Table 8 summarises the pay-out results from the in vivo trials with pulse-release capsules. It should be noted that the 6 replicates for each capsule design were staggered in terms of trial initiation date in vivo. For example, Trials N and R have capsules of the same orifice design and tablet arrangement. However, Trial N was initiated 4 days prior to Trial R. This was done in order to increase the chance of observing any unusual event during pay-out (e.g. capsule over-wetting or ‘volcano’-type co-extrusion). The data was analysed using Design Expert® software as a 2-factor DoE using orifice design and tablet arrangement as the two variables. The multi-hole design was found to significantly increase pay-out rate (p value<0.05), however, neither variable was found to effect linearity.

All trials ran to completion with no apparent signs of the ‘jamming’ which occurred in vitro (see Study 2 above).

TABLE 8
Pay-out summary results for trials with
capsules from Test Groups N, O, P and Q

| Test group | Mean Pay-out (mm/day) | Minimum R² | % CV |
|---|---|---|---|
| N | 1.011 | 0.993 | 0.22 |
| O | 1.198 | 0.992 | 0.74 |
| P | 0.991 | 0.989 | 3.56 |
| Q | 1.046 | 0.961 | 7.44 |
| R | 0.96 | 0.995 | 3.83 |
| S | 1.271 | 0.991 | 11.77 |
| T | 1.016 | 0.989 | 2.93 |
| U | 1.112 | 0.962 | 8.76 |

Table 8 illustrates the pay-out profiles of devices with the ‘pulse-dose’ tablet in the middle and end of the core. It can be seen that that the ‘pulse-dose’ tablets did not markedly effect the pay-out performance of the capsules with linear kinetics observed throughout the study. Table 8 shows the pay-out profiles of Test Group P and Test Group Q which contained the ‘pulse-dose’ tablets at the start, middle and end of the device. Linear kinetics were observed across the study period indicating that the three pulses did not markedly adversely affect the performance of the device.

Controlled release exit-dose capsules were assembled using three different orifice configurations. When a conventional single-hole orifice was employed, significant co-extrusion of the exit-dose tablet was observed to occur with the tablet in front. Importantly, this effect was markedly reduced when multi-hole orifices were used.

Controlled release pulsatile-dose capsules were assembled using three different orifice designs and two configurations of tablets. When tested in vitro, the pulse tablets tended to over-wet and temporarily jam the devices. The pulse tablets contain a lower viscosity grade of gel former and levamisole hydrochloride which is a soluble API. Consequently, they are likely to swell faster which may allow water up the walls of the device. Importantly, this phenomenon did not occur in vivo with any of the capsule designs studied and linear kinetics was observed through-out pay-out.

1. An intraruminal device, comprising:
an elongate body or body assembly substantially impervious to rumen fluids,
the body defining a barrel having a first end and a second end,
a dose of an active agent within the body to be accessible to rumen fluid via substantially only the first outlet,
a biasing arrangement within the body adapted to bias the active agent in the barrel towards the first end, and
a first outlet at the first end comprising two or more apertures in the outlet.

2. An intra-ruminal device of claim 1 wherein the outlet comprises a cap that seals the barrel.

3. (canceled)

4. An intra-ruminal device of claim 1 comprising 2 to 8 apertures.

5. An intra-ruminal device of claim 4, wherein the diameter of an aperture is about 1.0 to about 5.0 mm.

6. An intra-ruminal device of claim 4, wherein the total surface area of the apertures is within about 1 to about 40% of a typical single aperture cap.

7. An intra-ruminal device of claim 4, wherein the total surface area of the apertures is about 10.0 to about 60.0 mm2.

8. An intra-ruminal device of claim 4, wherein the apertures of the multiple-apertured caps are the same diameter.

9. An intra-ruminal device of claim 4, wherein the apertures of the multiple-apertured caps have a diameter that is within 2 to about 20% of their average size.

10. An intra-ruminal device of claim 4, wherein barrel and the cap are held together under heat and pressure.

11. An intra-ruminal device of claim 1, wherein the active agents are provided in a stack of tablets.

12. An intra-ruminal device of claim 11, wherein the stack of tablets include one or more controlled-release tablets.

13. An intra-ruminal device of claim 11, wherein the stack of tablets includes one or more exit dose tablets.

14. An intra-ruminal device of claim 11, wherein the stack of tablets includes one or more pulsatile release tablets.

15. A method of assembling an intra-ruminal device, the method comprising:
providing an intra-ruminal device comprising:
an elongate body or body assembly substantially impervious to rumen fluids, the body defining a barrel having a first end and a second end, and an opening at the first end,
at least one variable geometry device dependent from the body to assist rumen retention,
a dose of an active agent within the body to be accessible to rumen fluid via the first end,
a biasing arrangement within the body adapted to bias the active agent in the barrel towards the first end,
loading the active agent into the barrel,
attaching a cap located over the opening at the first end, the cap comprising an outlet in the top comprising two or more apertures.

16. (canceled)

17. A method of claim 15 having a pay-out (release rate) linearity of at least 0.940.

18. A method of claim 15 having a pay-out linearity measured over at least 80 to about 120 days.

19. A method of claim 15 having a pay-out (release rate) of about 0.30 to about 1.20 mm/day.

20. A method of claim 15, wherein at least about 80 to about 95% of each tablet extrudes before the next capsule in the stack of capsule begins to extrude.

21. A method of claim 15, wherein adjacent tablet in the stack of capsules exhibit a reduction in co-extrusion.
